(19) United States
(12) Patent Application Publication
(10) Pub. No.: US 2005/0079196 A1
(43) Pub. Date: Apr. 14, 2005

(54) Medical implant

(76) Inventors: Hans Henkes, Bochum (DE); Hermann Monstadt, Bochum (DE); Jurgen Speder, Bochum (DE); Ronald Kontek, Herne (DE)

Correspondence Address: FULBRIGHT AND JAWORSKI L L P;PATENT DOCKETING 29TH FLOOR, 865 SOUTH FIGUEROA STREET, LOS ANGELES, CA, 900172576, US

(21) Appl. No.: 10/494,013

(22) PCT Filed: Nov. 12, 2002
(86) PCT No.: PCT/EP02/12617

(30) Foreign Application Priority Data
Nov. 12, 2001 (DE) 101 55 191.6

(51) Int. Cl. A61F 2/54; A61F 2/00

(52) U.S. Cl. 424/423; 623/66.1

(57) ABSTRACT

The invention concerns a medical implant in the form of at least an elongated filament. The latter is preformed so as to have a specific structure when it is set on the implantation site. Said filament is traversed in the longitudinal direction by at least a removable retaining element which, until its removal, prevents the filament from having said specific structure.

The invention relates to a medical implant in the form of at least one elongated filament, said filament being preformed so as to have a superimposed structure which it assumes during implantation at the placement site.

The invention, furthermore, relates to a device for the implantation of such implants in body cavities and vessels.

It is known from prior art to treat vascoconstriction (stenoses) with the help of stents (vascular endoprostheses, vessel props) which are inserted into the stenotic area where they keep the vessel lumen open due to their inherent stiffness. It is further known to use such stents for closing off vessel wall ballooning (aneurysms) or fistulae.

For this purpose, balloon-dilatable stents were traditionally used. For placement these are crimped over a non-expanded balloon in non-dilated state, moved to the treatment location by means of a catheter system and then by expanding the balloon dilated and thus anchored within the vessel. Since there is no need for sophisticated supporting and guiding sheaths when placing balloon-dilatable stents in position these can also be inserted into very fine vessels. It is, however, problematic that on account of their plastic deformability they can easily be compressed when external pressure is exerted on them. Another disadvantage is encountered when anchoring such stents in that by applying high pressure they have to be expanded initially beyond the circumferential size they will finally have. Such an expansion beyond the circumferential size required may involve the risk of a vessel injury that may entail the formation of thrombs.

To rule out such risks it is known to apply self-expanding stents that are made of form-memory materials. These possess a braid-like structure and are initially introduced and moved in collapsed state through a catheter to the destination site where they expand either due to temperature changes (thermo-memory effect) or because the mechanical force exerted by the catheter (super-elasticity) is no longer effective. Such stents have the disadvantage in that the mechanisms required for their introduction are relatively expensive and space-consuming. (The known super-elastic expandable stents thus always require the use of a supporting and guiding sheath which necessitates a relatively large catheter size.)

For the introduction into small-lumen intra-cranial vessels it is thus known to use stents of shape-memory materials that initially are present in the form of an elongated filament, and not before they exit from the catheter will they assume the tubular structure of a stent due to the change in temperature or because of the compression force no longer being exerted by the catheter.

From DE 197 03 482 it is, for example, known for the treatment of aneurysms and similar diseases to use a stent consisting of two stretched out filaments that by the mechanical constraint of the catheter are kept, induced by tension, in that stretched out form until when being pushed out of the catheter said constraint is removed and they assume the actual form of a stent. For the first time, this enabled the use of stents having shape-memory properties also in vessels of very small lumen such as the intra-cranial and cerebral vessel branches.

These stents, nevertheless, suffer the disadvantage that they can be moved within the catheter only with difficulty because they necessarily exert from the inside pressure on the catheter inner walls. It is, moreover, necessary to exactly determine their destination site prior to pushing them out because they assume the stent shape as soon as they enter the blood vessel. Inside the vessel the stent can be moved only with difficulty. Furthermore, the expanded stent once having assumed its intended stent form can no longer be retracted into the catheter the inner lumen of which is necessarily of smaller dimension than the outer size of the stent because the catheter initially had to retain, only the much thinner sized, filament-like basic stent form in position.

Primarily similar problems are associated with other endovascular implants made of these materials.

In view of the disadvantages associated with the state of the art it is thus the object of the invention to provide implants that can also be introduced in vessels of small lumen and, moreover, are placeable without difficulty.

According to the invention this objective is reached by providing a medical implant of the kind first mentioned above which is characterized in that at least one retaining element passes through the filament's longitudinal axis, said element being removable from said filament and preventing the filament from assuming its superimposed structure before it is removed.

In this case, the filament preformed to assume a superimposed structure is kept under elastic tension or in a stress-induced martensitic state by action of the retaining element which is designed as a primarily stretched or elongated wire. Removing the retaining element from the filament will cause this constraint to be eliminated so that the filament is free to assume its predetermined superimposed structure. Due to the fact that the force is not exerted from the outside (for instance through the surrounding catheter) but from within the filament the implant according to the invention can be moved much better within the catheter and can even be repositioned when located external to the catheter until it has reached the ideal destination site in the organism.

In the following, the terms “proximal” and “distal” are understood in such a way that “proximal” refers to a point situated in a direction away from the target organism, that is towards the surgeon, whereas “distal” points to the destination site within the organism, i.e. away from the surgeon.

For the purpose of implantation the implant is preferably set upon an insertion aid and maneuvered towards the placement site using a micro-catheter. Insertion aid and retaining element are sized such that they can be separately manipulated by the surgeon. In their most simple form they are designed as straightforward linear elements that extend proximally through the micro-catheter up to the surgeon from which point the surgeon can manipulate them together with the micro-catheter. Having reached the destination site the implant according to the invention accommodating the retaining element is pushed out of the micro-catheter in distal direction and exactly placed in position, for example under radiographic observance. After the intended position has been reached the retaining element is removed so that the implant assumes the form prescribed by its superimposed structure. Even when the retaining element has been removed and assumed its prescribed form the implant may be repositioned by means of the insertion aid and, provided the lumen of the micro-catheter has been appropriately sized, even retracted into the catheter.

Basically, the intended shape of the implant determined by its superimposed structure can be freely selected to suit the respective purpose. For example, basket-shaped or tubular structures are particularly expedient for use when vascular malformations are to be occluded. In accordance with a preferred embodiment the development of the superimposed structure leads to the formation of a primarily tubular shape. In this case the implant can be employed, in particular, as stent. Preferably, the superimposed structure in this case constitutes a coil or spring, especially preferred is a spiral helix or helical spring. These structures exhibit the essentially tubular form which is typical of stents and, furthermore, are especially flexible and stable.

The sizing of the implants is governed by the destination vessel and may be easily determined by a responsible person skilled in the art. For an application in the fine intra-cranial or cerebral vessels implants having a coiled or spring structure are particularly suited, said implants having an outer diameter ranging between 0.5 and 10 mm.

It is especially expedient if the filament of the implant according to the invention possesses a recess arranged in the longitudinal axis which is intended to accommodate the retaining element. In this case the filament in its basic form is preferably a coil or helix whose lumen serving to accommodate the retaining element and, expediently, being closed at the distal end so that the retaining element is prevented from exiting there. In this case the filament itself constitutes a primary coil. The superimposed structure the filament turns into after the mechanical constraint exerted by the retaining element has been eliminated will then constitute the secondary structure (that is the secondary coil, for example). The retaining element is preferably loosely, but at least easily detachably, arranged in the recess of the filament so that it may be removed from the filament without difficulty. Especially expedient, because particularly atraumatic, is an embodiment featuring a filament that, particularly when provided as a primary coil/spring, has a round shape at the distal end.

Nevertheless, the filament may also have some other form, for example may be a profiled section, tube or have a folded form.

For the formation of the primary coil metallic wires are particularly suited that have a diameter ranging between 0.03 and 0.3 mm and, preferably, from 0.05 to 0.2 mm.

In accordance with an especially preferred embodiment the filament consists at least partially of a material that has shape-memory properties. An expedient material in this case is a metallic alloy capable of passing through a stress-induced martensitic transformation. Preferred in particular in this case are alloys that simultaneously are capable of passing through a temperature-induced martensitic transformation. For this purpose alloys containing titanium and nickel as well as iron and copper based alloys are especially suited.

The term “shape memory” is known to the above mentioned average person skilled in the art. It covers both the shape memory induced mechanically and that induced thermally. As materials having shape memory properties those materials are to be understood in the framework of the present invention that have either a thermal or a mechanical shape memory as well as materials having a thermally and mechanically induced shape memory.

Depending on temperature these materials are capable of changing their properties alternately between a more rigid and a very flexible state during which they pass through transitional states as well. They are much more resistant to bending and tension than traditional materials. Especially when in flexible state the material may be extremely bent and stretched out without suffering breakage. Only when the temperature has increased will the material again assume its rigid state which will require that it changes its form again when a preceding deformation has taken place. The relevant temperature threshold may be controlled or influenced in a way known to the responsible person skilled in the art via the composition of the material.

It is expedient if the implant according to the invention also contains a metallic alloy having shape-memory properties and, preferably, mainly consists of such material. These could be alloys which are only capable of undergoing a stress-induced martensitic transformation, but, preferably, such alloys are concerned that are capable of undergoing a stress-induced as well as temperature-induced martensitic transformation. For this purpose alloys containing titanium and nickel as well as iron and copper based alloys are especially suited.

Depending on temperature titanium-nickel alloys in this case show different crystal structures: The phase present at high temperatures is known as austenite. Their atomic configuration is cubic face-centered; this is the stable phase. At low temperatures, the atoms in such an alloy are of a tetragonally distored, body-centered cubic arrangement. This phase is known under the term of martensite. The martensitic phase which is governed by temperature is also termed temperature-induced martensite (TIM). By selecting a desired alloy composition it can be determined at which temperature a transition (transformation) from one phase to the other takes place, this may cover a range from −100 to 100° C.

If there is no external force present during the transformation from austenite to martensite (due to the temperature being reduced to below a critical value) no macroscopic change of shape can be observed. In its martensitic state the component may be easily deformed with a change of shape of up to 8% being achievable. As long as the material remains below its critical temperature threshold (the transformation temperature) the deformation is kept stable. However, if the deformed martensite is heated up the original shape is restored upon the transformation temperature being exceeded. This shape memory of the temperature-induced martensite which is influenced by an ambient temperature variation is also known as thermal shape memory.

Aside from this thermal shape memory metallic alloys may also possess a mechanical shape memory (super elasticity) which is associated with a stress-induced martensitic phase (SIM): In certain temperature ranges which may be varied by persons skilled in the art by selecting an appropriate alloy composition the transformation to the martensitic phase may also be effected mechanically by exerting an external force (stress-induced martensite). In this manner an expansion of up to 10% may be achieved. In the event the material remains at this temperature which is higher than the temperature threshold of the martensite to austenite transformation it will again return to its austenitic phase, i.e. an elastic recovery will occur.

A thermal transformation from martensite to austenite will, however, occur within a temperature range, not when a strictly limited temperature value is exceeded so that there are-transition phases in the material structure. If now the mechanical stress is eliminated that acts on a stress-induced martensite at a temperature within this transition range, a partial, stress-caused reconversion to austenite and thus a partial elastic recovery will take place. Only when a temperature increase is encountered will the transformation to the austenite phase be completed. In this case a combination of a stress-induced and temperature-induced phase transformation exists.

On account of the good technological properties these metallic alloys offer in their martensitic state (i.e. in the temperature- and/or stress-induced martenstic state) the material employed for the implant according to the invention is preferably selected such that the implant is kept in a stress-induced martensite state by the retaining element. Particularly preferred is the use of alloys offering both effects so that the filament passes through a mixed austenitic-martensitic transformation due to the increasing ambient temperature when leaving the catheter and being introduced into the blood vessel and because of the stress being eliminated when the retaining element has been removed.

To enable the mechanically induced shape memory to be advantageously used within the body alloys are especially suited that have a transformation temperature ranging between —15° C. and +38° C. and, in particular, between −15° C. and +20° C. For the utilization of the thermally induced shape memory within the body alloys having a transformation temperature of between +35 and +38° C. are particularly suited. The transformation temperatures especially suited to induce shape memory effects in the body, in particular mixed (stress and temperature included memory) effects as well, are sufficiently known to the competent person skilled in the art.

For the design of the retaining element any material is basically suitable that has adequate stability and tensile strength. The selection of the material and dimensioning of the diameter of the retaining element influence each other and, furthermore, depend on the material properties and diameter of the filament.

Details in this context are known to the compenent average skilled person so that suitable diameters and suitable materials may be determined and selected as necessary. In test performed by the inventors medical stainless steel wires of various diameters have proven their worth.

Particularly appropriate is an implant whose superimposed structure forms a coil the helix of which has loops of a pitch that varies over the length of the implant.

It is therefore particularly expedient for the closing off of aneurysms if, from the middle towards the ends, the pitches reduce so that there will be a denser arrangement in the middle and a looser arrangement of the helix loops at the ends. The implant with its dense middle section will then be deposited in front of the aneurysm. Moreover, this embodiment is especially expedient because the middle section of the implant has relatively high x-ray reflection characteristics and may thus serve as radiopaque marker.

In accordance with a preferred embodiment it is possible to coat the implant/the stent in a manner known per se with medically effective substances, for example with thrombosis inhibitory agents.

The invention, furthermore, relates to a device for the introduction of implants into body vessels and cavities with an implant in accordance with the above explanation and an insertion aid which is detachably connected to the proximal end of the implant.

It is especially advantageous in this case if the insertion aid is designed in the form of a tube (or some other linear element provided with a recess over its longitudinal axis), with the retaining element extending through the lumen of such tube from the implant in the proximal direction. It will be appropriate for the retaining element to be designed as an extension wire, particularly made of medical stainless steel.

Insertion aid and implant may be connected either directly or via a detaching module. Any connection that can be detached within the body is suitable. The use of a separate detaching module enables standard modules that can be easily produced- to be employed as insertion aid, implant and detaching module and, for that reason, is particularly cost-effective.

The device according to the invention is basically suited for any kind of implant detachment or severance, for instance for a mechanical, thermal or electrochemical detachment. These detachment or severance mechanisms and the pertinent technology are known to the competent skilled person. This also applies to the required design of the detachable connection between insertion aid and implant and of the severance element or other necessary elements of the device according to the invention.

As pe a preferred embodiment the device is designed for the electrochemical severance of the implant. For this purpose, it still comprises a catheter, a voltage source and a cathode. The catheter in this case is of electrically insulating design or the insertion aid itself is insulated, at least in its distal area (for instance, by means of a suitable coating or covering consisting of a shrunk-on sleeve). The implant will thus serve as anode and is arranged in the catheter so as to be slidable in longitudinal direction. The connection between implant and insertion aid has a location that is electrolytically corrodible so that when in contact with a body fluid the implant can be detached by electrolytic processes. As an alternative or additionally one or several severance locations may be arranged in the implant itself, for example equally spaced over the length of the implant, so that, to the extent the implant exits the catheter, one or several segments may be detached at the severance location arranged closest to the catheter. Such a configuration enables variable lengths of the implant to be disconnected with one or several detachment locations remaining inside the implant, or several implants to be deposited one after the other in the course of a treatment. This may offer benefits especially for the treatment of vascular malformations in places where bifurcations exist.

If the connection between insertion aid and implant is provided as a separate severance module it is particularly expedient for the severance module to be attached to the implant or the insertion aid by welding, soldering, bonding or mechanical joining processes. Especially beneficial in this case is a severance module that is provided with one proximal and one distal helix as well as a segment arranged in between that constitutes the electrolytically corrodible location.

The helixes of the severance module are permanently connected with the insertion aid on one side and with the implant on the other, preferably by welding, bonding or mechanical joining methods. After corrosion of the electrolytically corrodible location has taken place the distal helix with the implant is detached and thus placed in position. In this embodiment of the device according to the invention it is therefore expedient if the helixes of the severance module consist of material which is highly biocompatible and atraumatic, in particular a platinum alloy. The sizing of the severance module or of the helix forming part of the implant is selected such that it only represents a minimum length of the implant and in this way will not impede the placement process.

The electrolytic corrosion of the respective location will be ensured by selecting appropriate material combinations for the helixes and the segment arranged in between. These are adequately known to a person skilled in the art. In this context attention is drawn to publication WO 01/32085 A1 the disclosure content of which being expressly included herein.

Preferably, the sections or helixes of the severance module not capable of being electrolytically corrodible contain one or several of the following materials: Noble metals or noble metal alloys, corrosion-resistant ceramic materials, corrosion-resistant plastics, preferably platinum metal alloys (in particular Pt/Ir).

Also preferred is an embodiment of the device according to the invention whose detachable connection or severance module at the electrolytically corrodible location contains one or several of the following materials: Ceramic materials, plastics, non-precious metals or alloys of these metals, preferably stainless steel. In this connection, the stainless steel grades of type AISI 301, 303 or 316 and/or subgroups of these types are particularly suited. The ceramic materials and plastics employed for the design of the connection or severance module are electrically conductible.

In accordance with an advantageous embodiment of the invention combinations of materials are selected for forming the electrolytically non-corrodible helixes of the severance modules at the transitions to the electrolytically corrodible locations that are suitable for forming local elements. In this manner—and irrespective of whether the diameter at the corrodible points is reduced—the electrolytic detachment capability of the occlusion means is improved.

For this purpose material combinations are best suited which for the formation of the electrolytically corrodible locations make use of stainless steels, preferably of types AISI 301, 304, 316 or subgroups thereof, Ti or TiNi alloys or Co-based alloys with one or several of the following noble metals or noble metal alloys: Pt, Pt metals, Pt alloys, Au alloys or Sn alloys.

In another beneficial embodiment the end of the insertion aid is provided, for example, with a material coating of poor corrodibility or insulated with the aid of a shrunk-on sleeve coat so that it cannot be corroded electrolytically.

The device according to the invention is preferably intended for use in veterinary or human medicine and, more particularly, for the endovascular treatment of intracranial aneurysms and acquired or innate arteriovenous blood vessel malformations and/or fistulas and/or for the embolization of tumors by thrombozation. For this purpose the implant in its intended form is preferably designed as a stent, but may as well possess any other superimposed structure as may be expedient.

The invention is now described by way of examples as follows with reference being made to the figures showing the respective embodiments.

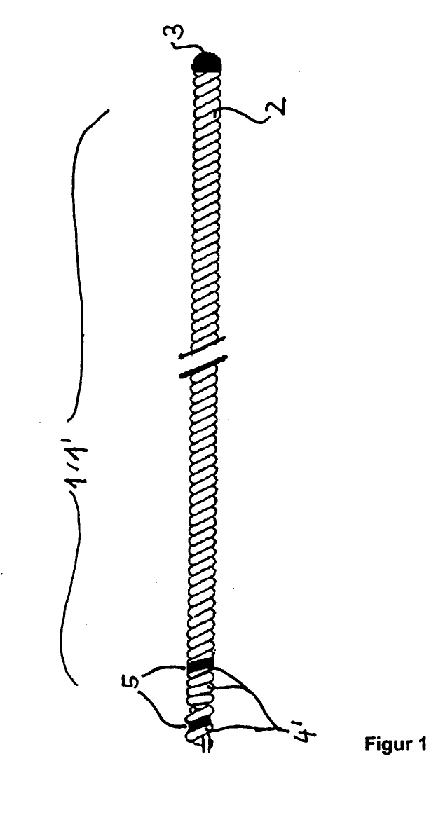

FIG. 1 is a side view of enlarged representation of an implant according to the invention having an elongated, filamentous form;

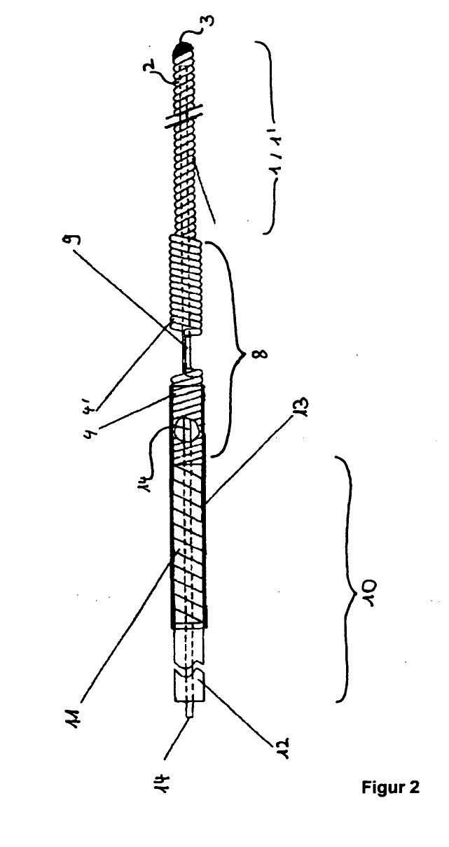

FIG. 2 illustrates also in an enlarged representation and as a side view the device according to the invention with elongated implant.

FIG. 1 shows in an enlarged side view a section of an implant 1 according to the invention. The distal end is situated on the right-hand side, the proximal end on the left-hand side of the drawing. In the implant 1 a retaining element designed as an extension wire of medical stainless steel is loosely arranged (non visible), said element keeping the implant 1 in its elongated form as filament 1′. The implant 1 is made of a wire 2 of a titanium-nickel alloy which possesses both mechanical and thermal shape memory characteristics.

The wire 2 has a diameter of 0.06 mm and has been wound so as to form a primary spiral helix having a diameter of 0.2 mm. At the distal end the spiral helix ends in a rounded tip 3 made of a platinum/iridium alloy. This design is particularly atraumatic. Simultaneously, tip 3 serves as a distal implant marker enabling the implant to be placed in position under radiographic observance. At its proximal end the filament 1′ is permanently welded at two seams 5 to the distal helix 4 of a severance module. In turn, the distal helix 4 is permanently welded to a thin wire 6 made of stainless steel which constitutes the electrolytically corrodible location.

FIG. 2 illustrates an example of another embodiment of the implant 1 according to the invention which in this figure is shown as part of an implantation device 7 according to the invention. The filament 1′ in this case as well has been designed as primary spiral helix made of a titanium-nickel wire 2 provided with a rounded distal tip 3 made of a Pt/Ir alloy. The severance module 8 connected on the proximal side to the filament 1′ comprises a proximal as well as a distal helix 4 and, respectively, 4′ made of Pt/Ir wire as well as an interposed segment 9 of medical stainless steel welded to the helixes 4/4′, said segment constituting the electrolytically corrodible location. The attachment of the severance module 8 on the proximal side is effected through its proximal helix 4 being connected by a welding method to the distal end of the insertion aid 10. In its distal area the insertion aid is designed as a wire spiral 11 (in this case as a flat-band spiral) and as a cannula tube 12 in its proximal area. The distal area of the insertion aid 10 as well as part of the severance module 8 are electrically insulated outwardly by means of shrunk-on sleeve 13.

The retaining element 14 designed as extension wire runs from the proximal end of the device to the distal area of the primary spiral of filament 1′ and thus traverses the insertion aid 10, the severance module 8 as well as the length of the filament 1′, as can be seen from its (partially) broken-line representation (the round cut-out in the proximal helix 4 does not form part of the device and is exclusively meant to improve the representation). The device 7 is maneuvered to the placement site in a micro-catheter. At the site the implant 1 as a stretched out filament is positioned in front of the aneurysm entry point by slidingly moving the insertion aid 10 within the blood vessel system. The extension wire 14 is also carried along.

The correct positioning is checked by radiographic observance of the tip marker 3 as well as the radiopaque helixes 4/4′ of the severance module 8. When the desired position has been reached the wire is removed by extracting it from the filament 1′ which, due to the fact that the retaining force is eliminated, causes the superimposed structure to form out, said structure in this case being a secondary spiral helix (not shown) serving as stent.

The spiral helix in this example is designed such that the density or closeness of its windings becomes less from the center towards the ends. This embodiment is especially appropriate for the occlusion of aneurysms. Furthermore, by way of the x-ray reflection caused by the more radiopaque middle section the correct placement of the implant can be verified during the operation even after the superimposed structure has formed. If the actually achieved positioning deviates from the optimum the placement of the implant may still be corrected with the help of the insertion aid 10. Even after the superimposed structure has formed the implant will still be flexible enough to be retracted into the catheter in a more or less elongated form so that, for example, in cases of wrong placements or faulty sizing of the superimposed structure the implant 1 may be completely removed from the blood vessel system.

When the fully shaped stent has been optimally positioned it will be detached from the insertion aid 10 by applying a voltage via a power source through electrolytic corrosion of the electrolytically corrodible location 9. The implant 1 serving as stent will then remain in the blood vessel causing the aneurysm to be occluded whereas the remainder of the device 7 is removed from the organism.

1. Medical implant in the form of at least one elongated filament, said filament being preformed so as to have a superimposed structure which it assumes during implantation at the placement site, characterized in that,
at least one retaining element (14) passes through the filament's (1′) longitudinal axis, said element being removable from said filament and preventing the filament (1′) from assuming its superimposed structure before it is removed.

2. The medical implant (1) according to claim 1, characterized in that the superimposed structure has a primarily tubular form.

3. The medical implant according to claim 2, characterized in that the superimposed structure assumes the form of a helix.

4. The medical implant (1) according to claim 3, characterized in that the helix has an outer diameter ranging between 0.5 and 10 mm.

5. The medical implant (1) according to any one of the above claims, characterized in that the filament (1′) is provided with a longitudinally arranged a recess for the purpose of accommodating the retaining element (14).

6. The medical implant (1) according to claim 5, characterized in that the filament (1′) is designed in the form of a helix.

7. The medical implant (1) according to claim 6, characterized in that the helix forming the filament (1′) has an outer diameter ranging between 0.1 and 0.5 mm.

8. The medical implant (1) according to claim 6, characterized in that the helix forming the filament (1′) consists of a wire (2) having a diameter ranging between 0.03 and 0.3 mm and preferably between 0.05 and 0.2 mm.

9. The medical implant (1) according to any one of the above claims, characterized in that the filament (1′) consists at least partially of a material having shape memory properties.

10. The medical implant (1) according to claim 9, characterized in that the material is a metallic alloy capable of passing through a stress-induced martensitic transformation.

11. The medical implant (1) according to claim 10, characterized in that the alloy is capable of passing through a temperature-induced martensitic transformation.

12. The medical implant (1) according to claim 9 or claim 10, characterized in that the alloy is an alloy containing titanium and nickel, an iron-based or copper-based alloy.

13. The medical implant (1) according to any one of the above claims, characterized in that the retaining element (14) is a metallic wire, preferably a wire consisting of medical stainless steel.

14. The medical implant (1) according to any one of the claims 3 to 13, characterized in that the pitches of the helix loops formed by the superimposed structure vary over the length of the implant (1).

15. The medical implant (1) according to claim 11, characterized in that the pitches reduce from the middle towards the ends.

16. The medical implant according to any one of the above claims, characterized by at least one severance module arranged therein provided with an electrolytically corrodible location.

17. The medical implant according to claim 16, characterized in that it is provided with severance modules arranged at regular intervals.

18. Device (7) for the placement of implants in body vessels and cavities with an implant (1) in accordance with any one of the above claims 1 to 17 and an insertion aid (10) which is detachably connected to the proximal end of the implant (1).

19. The device (7) according to claim 18, characterized in that the insertion aid (10) has the form of a tube, with the retaining element (14) extending through the lumen of such tube from the implant (1) in the proximal direction.

20. The device (7) according to claim 19, characterized in that the implant (1) and insertion aid (10) are interconnected by a severance module (8).

21. The device (7) according to any one of the claims 18 to 20 comprising a catheter, a voltage source and a cathode, with the implant (1) serving as anode and being longitudinally movable in the catheter, with the connection between implant (1) and insertion aid (10) having an electrolytically corrodible location (9) so that the implant (1) can be detached by electrolytic processes when in contact with a body fluid.

22. The device (7) according to claim 21, characterized in that the severance module (8) is provided with a proximal and a distal helix (4/4′) as well as a segment (9) arranged in between, with the helixes (4/4′) consisting of a material whose susceptibility to electrolytic corrosion is lower than that of the interposed segment (9).

23. The device (7) according to claim 22, characterized in that the severance module (8) is non-detachably connected to the implant (1) and the insertion aid (10) by welding, soldering, bonding or mechanical joining processes, particularly by force- or form-closing methods.

24. The device (7) according to any one of the claims 18 to 23, characterized in that the insertion aid (10) has, at least in part, the form of a helix or spring.

25. The device (7) according to any one of the claims 18 to 23, characterized in that the insertion aid (10) is surrounded, at least in part, by an electrically insulating shrunk-on sleeve or an electrically insulating coating.
